(19) United States
(12) Patent Application Publication
(10) Pub. No.: US 2008/0050769 A1
(43) Pub. Date: Feb. 28, 2008

(54) Method for detecting bioparticles

(75) Inventors: Jung-Tang Huang, Taipei (TW); Yu-Huan Lin, Taipei (TW); Shao-Yi Hou, Taipei (TW); Shiuh-Bin Fang, Taipei (TW)

Correspondence Address: Jung-Tang Huang, 5F., No.7, Lane 10, Sec. 2, Bade Rd., Da-an District, Taipei City, 106, TW

(73) Assignee: Jung-Tang Huang

(21) Appl. No.: 11/895,318

(22) Filed: Aug. 27, 2007

(30) Foreign Application Priority Data
Aug. 25, 2006 (TW) 095131439

(51) Int. Cl. C12Q 1/18; B01D 57/02

(52) U.S. Cl. 435/32; 204/450; 204/545

(57) ABSTRACT

This invention disclosed a method to detect bioparticles in the biological samples (stools, urine, or other body fluids). Bioparticles (e.g. virus, bacteria, and cells) often serve as carrier/indicator of pathogens and/or toxins. The method employs a substrate with interlaced comb-like electrodes on which a certain amount of sample mixed with antibodies-coated gold nanoparticles is dropped. Then the alternative signals with specific frequency bands are applied on the comb-like electrodes so that under such a DEP force the Au-modified bioparticles can be separated from the other constituents of the sample and can be absorbed effectively onto the edges of the electrodes. After rinsed with water to remove the residual sample several times, the device will be measured for the impedance of the absorbed bioparticles on the edges of the electrodes. The measured impedance deviation in comparison with that of the reference empty comb-like electrodes will quantify the amount of the absorbed bioparticles.

### TECHNICAL FIELD

A bioparticles detection method using dielectrophoresis (DEP) force that is created by interlaced comb-like electrodes on a chip can target bioparticles, which are mixed with and then attached to the corresponding antibodies-coated gold-nanoparticles. After the implementation of collection and capacitance measurement, the bioparticles can be quantitatively detected.

### BACKGROUND OF THE INVENTION

Bioparticles including viruses, bacteria and other cells, often serve as pathogens or toxic carriers/indicators. Due to the increasing prevalence of infectious diseases in early era, the first choice of treatment is use of antibiotics. Antibiotics were accidentally found in the culture of bacteria by the British scientists Franz in 1928. This significant discovery benefits the future patients with various infectious diseases. However, development of antibiotic resistance has become a big problem and confused clinical doctors a lot. Appropriate antibiotics with a good efficacy of inhibiting or killing the pathogens should be decided when the antibiotic therapy is started. Because the current methods of detecting pathogens and antibiotic resistance are mostly time-consuming, the golden time of choosing a proper antibiotic for medical treatment is usually delayed. This invention uses Salmonella as an objective of embodiment, aims at shortening the time to detect pathogens and to demonstrate characteristics of antibiotic-resistant pathogens. However, the invented method can be easily applied to other pathogens and biological particles.

Traditional detection methods of Salmonella include six stages: pre-enrichment, selective enrichment, chromogenic medium, identification of biochemical characteristics, and serum screening test. It needs at least 3-5 days before we know whether the pathogens grow and whether antibiotic resistance exists. Since this kind of detection method is time-consuming, there are many rapid detection methods of Salmonella have been developed and commercialized. The following classifications outlined are:

Improved selective medium: the traditional way to detect Salmonella needs to first vaccination proliferation in culture medium, then at least three different selective screening media was used to culture Salmonella from suspicious colonies. Due to their complicated and time-consuming manipulations, the market is flooded with many advertised and more specific biochemical selective media, such as MSRV, SMID, MLCB agar, and Rambach agar, but these medium may have some problems of false positivity. In order to identify whether the grown colonies are truly or falsely positive, we need further follow-up experiments for confirmation.

Biochemical identification kit: biochemical identification of colony requires preparation of the various media and reagents, which consume too much time and manpower, hence, there are many commercial kits for detecting Salmonella, such as API 20E, MICRO-ID, Enterotube II, and Enterobacteriaceae Set II. Four sets of the above group have been recognized by AOAC Association of the United States.

Immunosorbent assay: the use of antigen and antibody with high specificity and high affinity characteristics. Its biggest advantage is easy to use. Anyone can use it according to the manual with no need of expensive equipments to get results in a short time. At present, there are some commercial rapid detection of Salmonella kits, such as 1-2 test, TECRA, Salmonella-Tek, Reveal, Assurance Gold, (VIP) Visual immunoprecipitate assay, and LUMAC P ATH-ATIK etc.

DNA testing method: using unique microbial genes (DNA) to develop the detection method for identifying Salmonella. At present, there are some commercial kits, such as the GENE-TRAKR-DNAH, BASR, and TaqManR.

Automation equipment: mini VIDAS is an automated ELISA analysis, used to produce fluorescent substrates of the enzymes. It can be used to rapidly screen Salmonella. The Salmonella detection kit used in this equipment has been recognized by FDA.

All of the above methods were designed for speeding up the detection process, but they still take a few days to know the results that is apparently not enough for emergency. Therefore, the invention developed a novel detection method to get the existence of pathogens and quantities of biomaterials in dozens of minutes to a few hours, which can assist physicians in rapidly diagnosing Salmonella infections and choosing appropriate antibiotics in an early time.

### SUMMARY

The first aspect of the present invention is to provide a method that uses specificity between antigen and antibody to make antibody-coated metal nanoparticles to attach to the target bio-particles, and change their original dielectric properties to achieve the purposes of the pathogen collection.

In the further aspect, the present invention provide a chip to collect the biological particles and measure the changes of capacitance so that it can rapidly learn the quantities of the biological particles.

The further aspect of the present invention is to improve existing bacteria or virus detection technologies by fastening and simplifying the course of detection and laboratory works to obtain the measured results. There is no limitation of performance in users' experiences and laboratory facilities.

The another aspect of the present invention is to provide clinical doctors with a testing method for antibiotic resistance of the collected pathogens that make them start antibiotic treatment in patients quickly, properly and accurately.

### BRIEF DESCRIPTION OF DRAWINGS

The invention may best be understood by reference to the following description taken in conjunction with the accompanying drawings that illustrate specific embodiments of the present invention.

FIG. 1 illustrates the biological particles detection chip of this invention (a) assembly diagram, (b) exploration diagram.

FIG. 2 illustrates the gold-nanoparticles using chemical methods to connect to antibodies, which can be attached to the surface antigen of biological particles through specificity between antigen and antibody.

FIG. 3, illustrates that nano-gold modified particles can be modeled as simplified biological cell core and simplified biological cell membrane, furthermore the cell and gold layer are integrated into a homogeneous sphere.

FIG. 4 is a diagram shows the DEP strength as a function of conductivity of solution contained unmodified cells and applied field frequency.

FIG. 5 is a diagram shows the DEP strength as a function of conductivity of solution contained non-Au modified cells and applied field frequency.

FIG. 6 illustrates the flow chart of the chip of the present invention fabricated by injection compression molding.

FIG. 7 depicts the flow chart of fabricating the shadow mask for sputtering or evaporating.

FIG. 8 illustrates flow chart of MEMS fabrication process for chip of present invention.

FIG. 9 shows the experimental results of dielectrophoresis for modified and unmodified Salmonella.

### DETAILED DESCRIPTION

The invention is based on a mechanism of dielectrophoresis to manipulate the biological particles. Proposed by H. A. Phol in 1978, dielectrophoresis is a phenomenon that describes polarized particles with dielectric properties, in an alternating electric field of appropriate size, can induce electric dipole. This interaction with the irregular external electric field will enable the particles move to in the direction of larger or smaller field.

The invention uses gold nanoparticles to modify the surfaces of biological particles such that the small difference of physical characteristics between different bio-particles can be amplified and separated from each other in a short time. In other words employing dielectrophoresis to directly manipulate purification of the sample, which can spare the chemical test, shorten test time, and enhance the efficiency of pathogen detection.

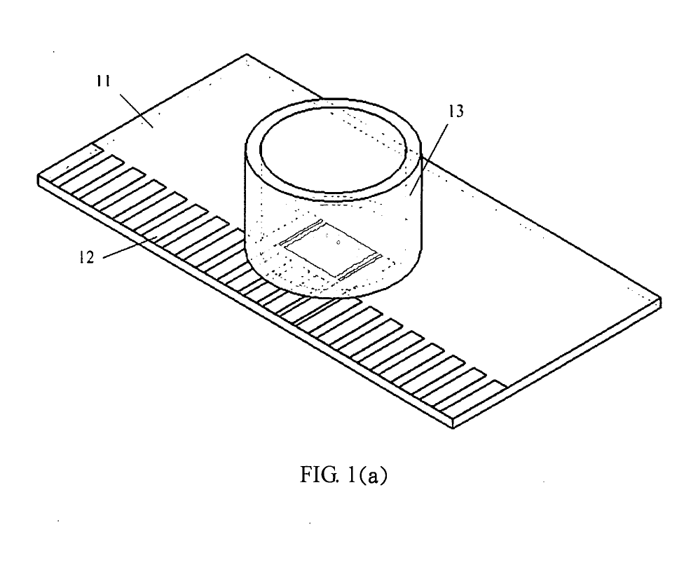

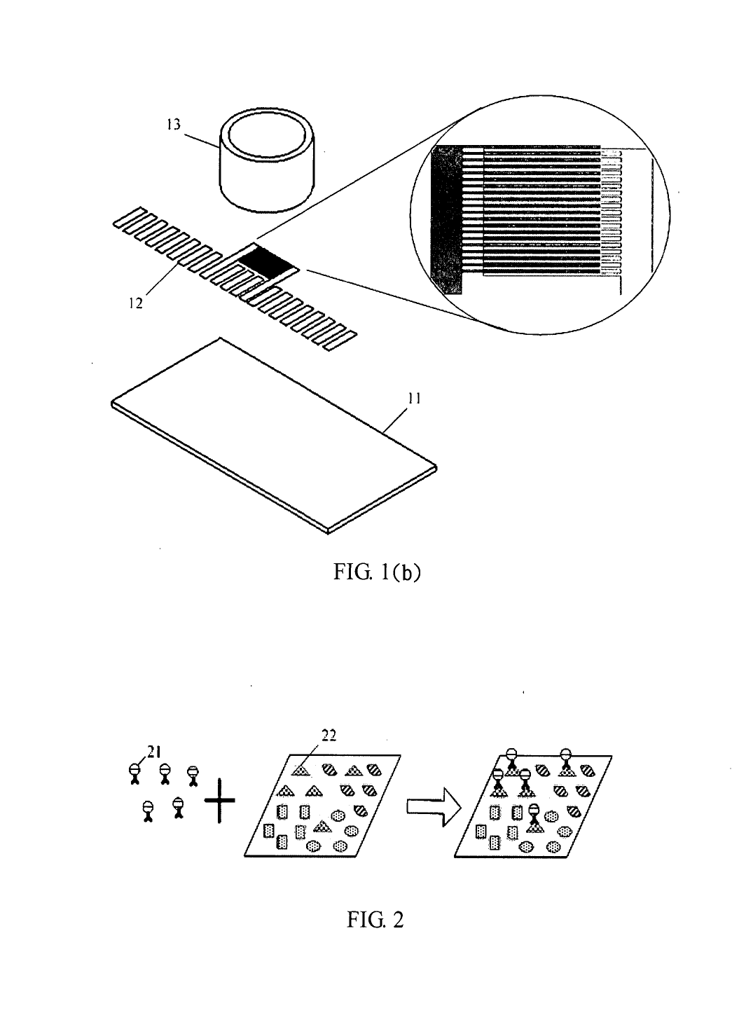

The biological particles detection chip of this invention is shown as FIG. 1. The main components of the chip include: (1) substrate 11; (2) electrodes 12; (3) cavity 13. Chip size is as large as the size of glass slide. Electrodes 12 with thickness of about 0.35 μm, are placed on the top of substrate 11. In addition, on top of electrodes 12 a storage cavity 13 is set for confining fluid. The injected biological particles in the fluid can respond to the signal of function generator applied on the electrodes 12. With signals of specific frequency applied to electrodes 12, dielectrophoresis force can be induced to implement the follow-up collection and observation.

The general dielectrophoresis force can be explained as interaction between electric field \vec{E}(t) and induction coupling moment \vec{m}(t), which can be simplified as indicated in Equation (1):
\vec{F}(t)=(\vec{m}(t)·∇)\vec{E}(t) (1)
Where \vec{m}(t) is from Maxwell-Wanger theory:
\vec{m}(w)=4π∈ₘr³[f_{CM}]\vec{E}(w) (2)
∈ₘ is the dielectric coefficient of suspending medium, ∈ₚ is the dielectric coefficient of particle in the medium, due to the induction coupling moment is related to angular frequency, f_{CM} also called as polarization factor or Clausius-Mossotti factor, defined as:

$${f}_{CM} ( {ɛ}_{p}^{*} , {ɛ}_{m}^{*} ) = \frac{{ɛ}_{p}^{*} - {ɛ}_{m}^{*}}{{ɛ}_{p}^{*} + 2 {ɛ}_{m}^{*}} \qquad ( 3 )$$

where ∈* is complex form of dielectric coefficient, having relationship among dielectric coefficient (∈), conductivity (σ), and applied electric field frequency (ω), described as
∈*=∈−iσ/ω (4)

From the above equations (1)-4), under a generalized and time-averaged electric field one can derive the traditional dielectrophoresis force, F_{dep}:
\vec{F}=2πr³∈ₘRe[f_{CM}]∇(Eᵣₘₛ²) (5)

From the equation (5), it is not difficult to discover that dielectrophoresis force is directly related to size of the particles as well as the gradient of the square root-mean-square value of the electric field versus location. The sign of DEP force also depends on the sign of real part of polarization factor f_{CM} Therefore, we can change the conductivity of solution, the frequency and the electric field distribution to control the behavior of suspended particulates in the solution.

The use of dielectrophoresis to tell distinction among biological particles depends on the dielectric properties of biological particles. But when biological particles have the similar dielectric characteristics in the general frequency range, it is difficult to distinguish among them. Using nano-metals (in the following only gold nanoparticle is described as an example) to modify biological particles can increase the differences of their effective dielectric characteristics. As shown in FIG. 2, the gold nanoparticles using chemical methods to connect to antibodies 11, can be quickly attached to the surface of biological particles through specificity between antigen 12 and antibody 11. Thus the surfaces of the biological particles are coated with a layer of gold nanoparticles.

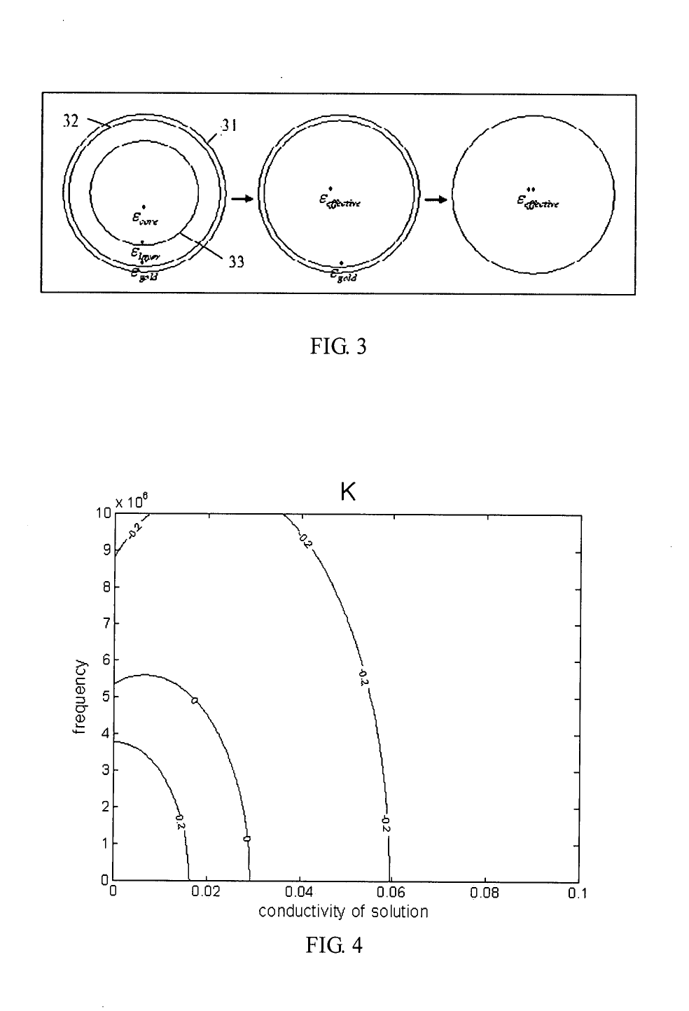

Gold-nanoparticles have good conductivity which makes the surface conductivity of biological particles be greatly enhanced. According to shell theory, as shown in FIG. 3, biological core 23 and biological cell membrane 22, can be simplified in effective cell, then the effective cell and gold layer 21 are further simplified into a homogeneous sphere. Employing shell theory will simplify the gold-nanoparticles modified bio-particle into a uniform ball. Assume dielectric coefficient of outer ring as ∈ₘ, inner dielectric coefficient as ∈ₚ, and substitute into the thin shell theory formula (6)

$${\underline{ɛ}}_{m} = {ɛ}_{m} \{ \frac{{a}^{3} + 2 ( \frac{{\underline{ɛ}}_{p} - {\underline{ɛ}}_{m}}{{\underline{ɛ}}_{p} + 2 {\underline{ɛ}}_{m}} )}{{a}^{3} - ( \frac{{\underline{ɛ}}_{p} - {\underline{ɛ}}_{m}}{{\underline{ɛ}}_{p} + 2 {\underline{ɛ}}_{m}} )} \} \qquad ( 6 )$$

where ∈ₚ* the complex dielectric coefficient of biological particles in equation (7), and ∈ₘ* complex dielectric coefficient of the outer layer of gold particles in equation (8),
∈ₚ*=∈ₚ−iσₚ/ω (7)
∈ₘ*=∈ₘ−iσₘ/ω (8)

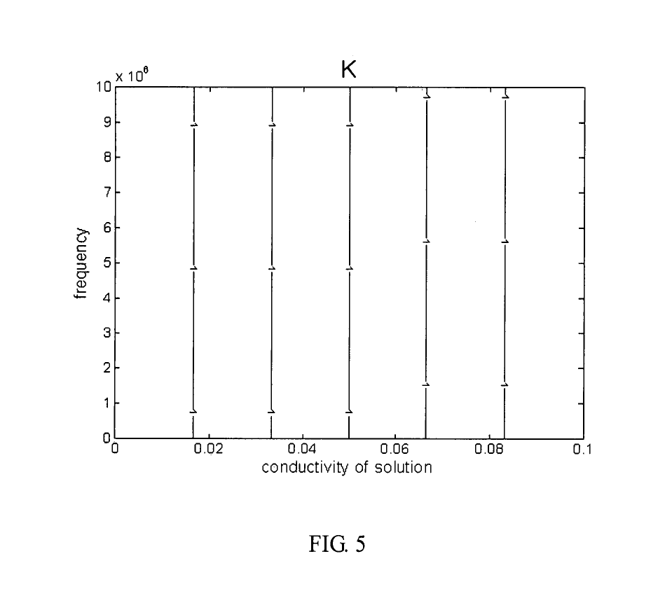

Substituting into the equation (6), we can find the key factors of affecting positive and negative polarization depends on the conductivity and frequency. Using MATLAB to calculate formula of corresponding frequency to conductivity of the solution, the higher the conductivity in the case the more easily the negative DEP happens, and the original unmodified cells with nano-particles can produce negative DEP phenomenon in the low-frequency range as shown in FIG. 4. The relation between solution conductivity of cells unmodified with gold nanoparticles and frequency, “-0-” means zero-cross line, below this line is negative DEP; and above this line is positive DEP. But after modification with gold nanoparticles the biological particles produce positive DEP only as shown in FIG. 5, from 0 to 10 MHz, which indicates the modified biological particles can be separated from unmodified ones in the low frequency range.

Preparation of Antibody-Coated Gold Nanoparticles:

Because gold nanoparticles have good affinity for effect of the object surface modification, they are used to modify the surface properties of biological particles. Common nanoparticles preparation methods include laser ablation method; metal vapor synthesis method such as vapor liquid solid growth, physical vapor deposition, chemical vapor deposition; and chemical reduction method such as salt reduction, electrochemical, sonochemical preparation, and seed-mediated growth.

Mix received 400 μl gold nanoparticles to 100 μl of 0.26 mM K₂CO₃. Add 1 μl antibody to a solution then mixing. Add 150 μl of 5% BSA solution to cover the location of gold nanoparticles where no antibody is bonded with, under 4° C. and 6,000 g centrifugal for 25 minutes. Carefully take away the supernatant, and then add 1×PBS to yield a total volume of 20 μl.

Chip Manufacturing Injection-Compression Molding

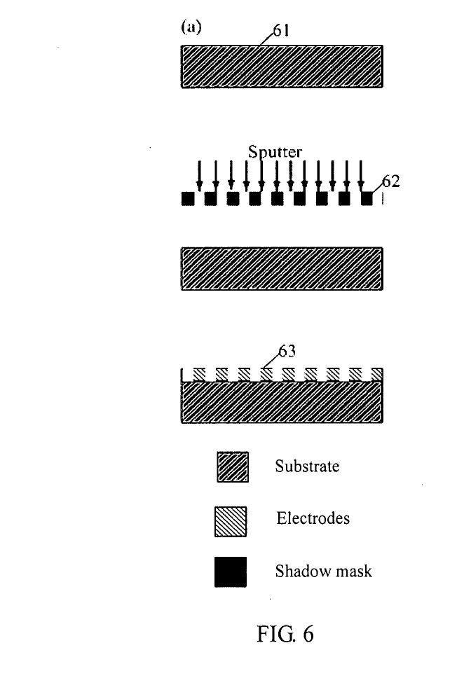

Step 1: Referring to FIG. 6 (a), fabricate the plastic chip 61 made of transparent material, such as polycarbonate (PC) by injection-compression molding technology. The chip includes a reactor structure (not shown in the figure). Clean the surface of the chip.

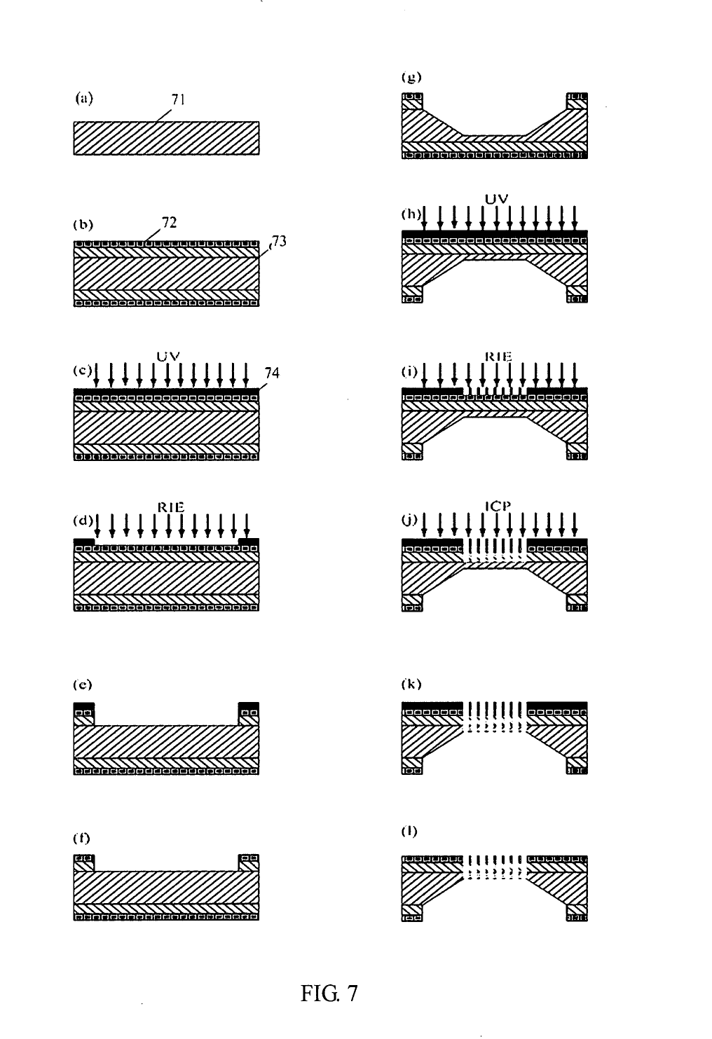

Step 2: Use anisotropic-etching with lithography exposure technology, and Inductively Coupled Plasma (ICP) etching to fabricate shadow mask. As shown in FIG. 7 (a)˜(g), first use standard lithography process to define pattern on the silicon substrate 71, then by reactive ion etching (RIE) and KOH to etch out an membrane structure with aim to reach micron precision and resolution. In this phase of wet etching a back layer can be reserved to provide the diaphragm with adequate mechanical support for the benefit of the follow-up mask manufacturing process. Then again behind the diaphragm structure use standard photolithography process to define the required electrode patterns, and employ reactive ion etching (RIE) and ICP to etch through, and then remove unnecessary PR to complete the shadow mask, as shown in FIG. 7 (h)˜(l).

Step 3: Referring to FIG. 6 (b), use the shadow mask 62 to cover the chip's reactor.

Step 4: Referring to FIG. 6 (c), sputter or evaporate the patterns of interdigited comb-like electrodes and the associated connection wiring 63 to complete the production of single-chip.

Fabrication of MEMS Process

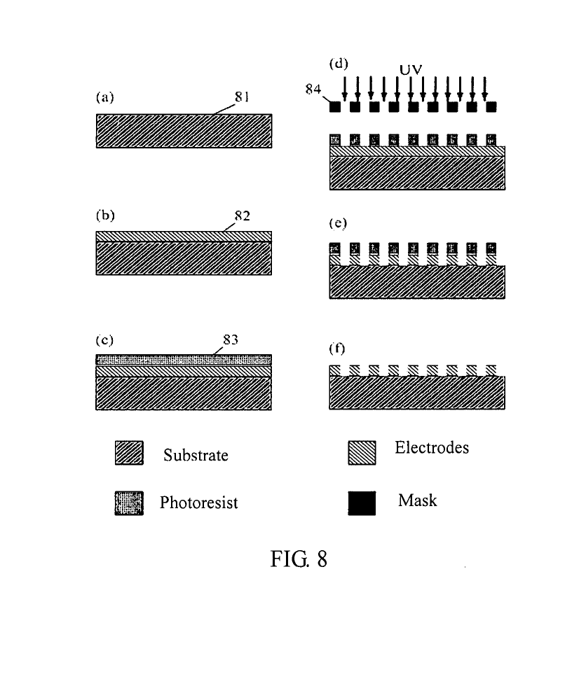

Step 1: Referring to FIG. 8 (a), cleanse the glass substrate 81.

Step 2: Referring to FIG. 8 (b), deposit metal layer as detection electrode 82 on the substrate with thermal evaporation.

Step 3: Referring to FIG. 8 (c), spin coating photoresist 83 on the aluminum layer.

Step 4: Referring to FIG. 8 (d), pattern the photoresists with mask 84 to define the interdigited comb-like electrodes.

Step 5: Referring to FIG. 8 (e), strip the undesired photoresists and implement the wet etching of the metal layer to complete the pattern transfer.

Step 6: Referring to FIG. 8 (f), remove the remaining photoresists to complete the chip fabrication.

Traditionally, DEP can be used to separate pathogens. At the same conductivity of solution, most pathogens have different dielectric properties. Pathogens will be manipulated by the positive or negative DEP and the magnitude of DEP, indicating that they can be isolated or collected or even counted. However, they all are modeled as a homogeneous sphere by neglecting the original cell cytoplasm, the presence of the membrane. Instead, here we consider the above cell structure and use the single-shell model to more approximate the real characteristics of pathogens, including Salmonella, Escherichia coli, Staphylococcus aureus, Pseudomonas aeruginosa, Malaria original bacteria, and leukopenia, etc. In addition, if nanoscale particles of metal are bonded with antibodies for pathogens, they can be connected with the pathogens and their original dielectric properties change accordingly as shown in the following table. Positive DEP indicates Salmonella will be adsorbed to electrodes; the negative DEP will expel the Salmonella away from electrodes. There is larger difference of DEP for gold nanoparticles. Under specific conductivity of solution, the current invention effectively uses different combinations of frequencies and voltages to isolate the pathogen from biological samples.

frequency (Hz)

| | <5k | 5k-1M | 1M-10M | >10M |
|---|---|---|---|---|
| unmodified | Negative | Positive | Positive | Positive |
| | DEP | DEP | DEP | DEP |
| modified | Positive | Positive | Positive | Positive |
| | DEP | DEP | DEP | DEP |

Testing Methods

Step 1: Prepare solution containing gold nanoparticles attached by various antibodies. The concentration can be diluted as required, which basically prepare as rate of tenth fold of reference concentration.

Step 2: Mix samples containing pathogens with the appropriate concentration of the antibody-coated gold nanoparticles into solution. Leave for a period of time to make antibody-coated gold nanoparticles be fully integrated with pathogens. Here the appropriate concentration of the sample can refer to the one for full pathogens and antibody-coated gold nanoparticles combination. Basically, the smaller concentration is better.

Step 3: Draw a certain amount of the mixture, half of which is diluted into one-tenth concentration. Drip the two halves of mixture to the A and B wells respectively, impose specific frequency signals to comb-shaped electrodes for a few minutes to implement DEP separation. Note that the concentration of A well is 10 times that of B well, so we can see whether the saturation occurs. Get rid of the supernatant and fill with conductive water.

Step 4: Use a lock-in amplifier while implementing DEP, through the impedance analyzer to measure capacitance.

Step 5: As the electrode of the chip and the amount of space has a certain limit size, the adsorption volume of pathogens has limitations. This will be reflected in the capacitance measurement, if both A and B wells are saturated, indicating the pathogens have very large quantity. If not, the correct concentration of pathogens can be obtained.

Basic Test and Analysis

Salmonella is a group of small, gram-negative organisms, which can produce gas without fermentation of lactose. Its growth temperature is 5.3-46.2° C., the optimum temperature is 35-37° C. and pH range between 4-9. It is frozen-resistance in water. It exists in animals' intestines, through the people, dogs, cockroaches, rodents and other paths to contaminate food products. Salmonella is one of the bacteria that can lead to food poisoning and enterocolitis. Only a small amount of oral inoculum (<10⁵ cells) of Salmonella will cause disease. Salmonellosis can be divided into three categories. The first is Salmonella typhi caused typhoid fever. This is the most serious one Salmonella food poisoning symptoms; The second category is by Salmonella paratyphi A, B, C induced disease paratyphoid, more moderate symptoms; The third category is so called nontyphoid Salmonellosis induced mostly by Salmonella typhimurium and Salmonella chaleraesuis. Salmonella enteritidis induced gastroenteritis, symptoms of vomiting, diarrhea and abdominal pain.

The embodiment employed Salmonella enteritidis from a patient in the hospital, and develop new Salmonella samples, mixed in KCL solution for detection. Salmonella is whipped up a small part from the dishes and immersed in the KCL deionized water (1 mg/3 ml) with conductivity of the 2 μS/cm. Stay still for three hours and stain them. In addition, redeploy a group of Salmonella samples and add the antibody-coated gold nanoparticles to wait for three hours until complete bonding is achieved, and then stain them.

Cleanse the two groups of chips by using deionized water (DI Water) to remove impurities on the surface, and dry residual water on the chip with nitrogen. Use micro-titration to drop the sample solution on the finished electrodes of the chip, and cover with glass to prevent interference of other factors (such as air flow and moisture evaporation).

Control Group: Unmodified Salmonella

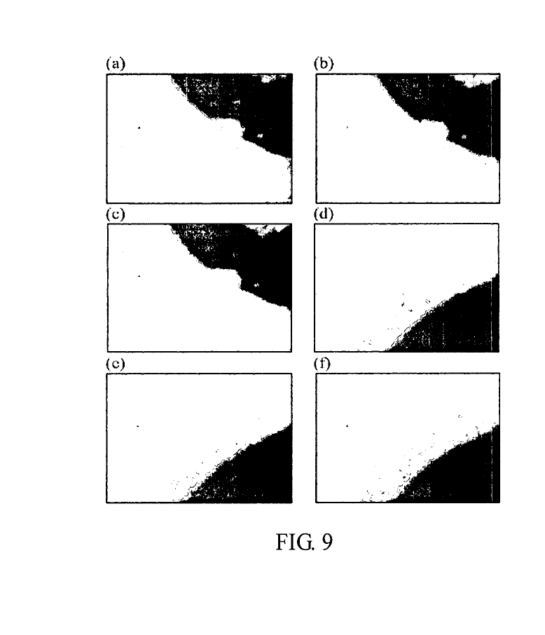

Some of solution samples is dropped by micro-titration on the electrodes of the chip, and covered with glass. FIG. 9 (a) shows the particle distribution before the electric field is imposed. Following the applied electric field of 10 MHz, Salmonella in solution with conductivity of the 2 μS/cm is adsorbed on the electrode by dielectrophoresis force, as illustrated in FIG. 9 (b). The original randomly distributed Salmonella was polarized by the effects of electric field and aligned along the direction of electric field extending several layer surrounding the electrode. When the electric field frequency gradually reduced, the dielectrophoresis force on Salmonella became weaker accordingly; the Salmonella adsorbed on the electrode were also reduced. When the electric field frequency was turned down to the vicinity of 5 kHz Salmonella conducted by negative dielectrophoresis left electrode. As shown in FIG. 9 (c) the Salmonella originally attached to the electrode were instantly expelled. Again if the electric field frequency is restored to more than 5 KHz, several layers of Salmonella are adsorbed around the electrode.

Experimental Group: Modified Salmonella

Following antibody-coated gold nanoparticles can have sufficient time to attach Salmonella, the chip with produced electrodes will be dropped the solution by micro-titration and covered with glass. Before applying electric field the gold-nanoparticles modified Salmonella shows the similar situation as unmodified one. When imposing the electric field of 10 MHz, Salmonella connected to the antibody-coated gold-nanoparticles is still conducted by dielectrophoresis force to adsorb on the electrode. As shown in FIG. 9 (d), once the frequency is lowered and the quantity of Salmonella absorbed by attraction electrode decrease as the weakening of the forces. When the frequency of electric field is instantaneously changed to 5 kHz, as shown in FIG. 9 (e), Salmonella connected to the antibody-coated gold-nanoparticles is still exerted by positive dielectrophoresis and adsorbed on the electrode. Due to decrease of the frequency, electric field strength reduced, which leads to adsorbed on the electrode to reduction of sample layers. But it can be observed that there are still some Salmonella adsorbed on the electrode. Once the electric field is stopped, Salmonella as shown in FIG. 9 (f) gradually desorbs from the electrode back to the original disorder.

From the above results we can clearly realize that the modified Salmonella has changed its original dielectric properties. When imposed the signal of 5 kHz frequency, Salmonella is conducted by the negative DEP force to leave away electrodes. On the contrary, modified Salmonella is exerted by positive DEP forces to continuously adsorb onto the electrode. Making use of this characteristic can achieve the purpose in separation of Salmonella from other bacteria. According to this simple cell surface modification, any cells or pathogens can change their dielectric properties, as long as the bioparticle has its surface antigen, which can be combined with the corresponding antibodies and nano-particles. Apart from the use of gold-nanoparticles, the nano-particles can also be replaced by alternative metal nanoparticles, as long as its nature and stability allow the binding of antibody. Besides, it can also combine magnetic beads with antibodies to modify cells so the cells can be purified and collected in the external magnetic field. Following the isolated target cells can be processed for further analysis such as counting, concentration measurement, antibiotic-resistance testing. As a good example in our novel model, Salmonella can be isolated quickly from the stool samples and then antibiotic resistance can be detected. Physicians will have a better understanding regarding the infection levels of the specific pathogen in patients and provide them the appropriate antibiotics.

The application of nano-magnetic beads is further described below. The chip has non-magnetic comb-shaped electrodes, on which at least one reacting well is set. The implementation steps include: (a) Add samples and nano-magnetic antibodies, which are specific to the target pathogens, in the test tube. Mix them in an aqueous solution so that the target pathogens can bind with the corresponding antibodies-coated nano-magnetic beads; (b) Take out a fixed amount of mixture, and drop into the small storage tank on the chip; (c) Use an external magnetic field to adsorb and accumulate the pathogens combined with nano-magnetic antibody on the chip; (d) Dump out the supernatant mixture and add water, gently and repeatedly, until the removal of unnecessary residues in the samples to purify pathogens. During the performance, the external magnetic field remains to work for keeping the nanobeads-modified pathogens being absorbed on the chip; (e) Turn off the external magnetic field, and apply on the comb-shaped electrode with a specific frequency AC signal to continue adsorbing antibody-coated nano-magnetic beads combined pathogens. Connect the comb-shaped electrode with a lock-in amplifier and an impedance analyzer to measure impedance between the electrodes, particularly the capacitance, and then compare it with that from the blank comb-shaped electrode. The differences in measured values yield pathogens quantity adsorbed on the electrodes.

### EMBODIMENT 1

Directly collect 2 grams of the stool from patients with Salmonella to the conductive fluid of 10 ml, and mix with Salmonella antibody nano-gold for 30 minutes to ensure that the antibody-coated gold nanoparticles have fully integrated with Salmonella. Take out 0.1 ml of the above mixture and drop it in the chip of the present invention, and then apply signals of specific frequency to the comb-shaped electrodes for five minutes. The bottom of the device can further be heated so that it has mild solution convection to increase adsorption opportunities between the attached gold-nanoparticles and comb-shaped electrodes. Take out the supernatant of the mixture and add deionized water to rinse and keep purified adsorbing in the edge of comb-shape electrodes. Impose the comb-shaped electrode with a specific frequency AC signal to continue adsorbing modified pathogens. Connect the comb-shaped electrode with a lock-in amplifier and an impedance analyzer to measure impedance between the electrodes, particularly the capacitance, and compare it with that from the blank comb-shaped electrode. The differences in measured values reflect pathogens quantity adsorbed on the electrodes.

### EMBODIMENT 2

Antibiotics can be divided into two categories: first, bacteriostatic such as chloramphenicol, which hint protein synthesis but germs continue hyperplasia after removal of it; second, bactericidal such as penicillin and congeners, which inhibit the cell expansion but eventually lyse the cell wall and lead to cell death because of increased osmotic pressure inside the cells caused by their consecutive synthesis in the cytosol. When these two mechanisms of antibiotics fail, antibiotic resistance ensues.

Directly collect 2 grams of the stool from patients with Salmonella to the conductive fluid of 10 ml, and mix with antibody-coated gold nanoparticles until 30 minutes to ensure that the antibody-coated gold nanoparticles have fully integrated with Salmonella. Take out 0.1 ml of the mixture, drop it in the chip of the present invention, and apply signals of specific frequency to the comb-shaped electrodes for five minutes. The bottom of the chip can be further heated so that the solution has mild convection to increase adsorption opportunities between the Salmonella bound gold-nanoparticles and comb-shaped electrodes. Take out the supernatant of the mixture and add deionized water for rinsing and keeping purified Salmonella adsorbed on the edges of comb-shape electrodes. Apply comb-shaped electrode with a specific frequency AC signal to continue adsorbing the modified pathogens. Measure the impedance between the electrodes, particularly the capacitance, and compare it with that from the blank comb-shaped electrode. The differences in measured values yield pathogens quantity adsorbed on the electrodes.

Case (a) Bactericidal Antibiotics

Add a certain amount of bactericidal antibiotic to reduce Salmonella survival while it can also be optionally added with a buffer to adjust its pH value or concentration of magnesium ion (Mg++), etc, (See: W. G. Clark; D. C. Brater; A. R. Johnson; A. Goth “Goth's Medical Pharmacology” St. Louis: Mosby-Year Book, 1992). Measure the present impedance value, wait for a certain period of time to allow antibiotic reaction to occur thoroughly, and then measure the impedance value again. Compare the measured result with that without antibiotics to see if the numerical trend remains nearly unchanged or the reduction trend is a natural death. In this case, it shows that the antibiotic in unable to kill Salmonella. On the contrary, the antibiotic can kill Salmonella. Through this way it can detect the required dosage of the antibiotic and the antibiotic resistance of Salmonella. Notice that the culture medium is not used here to increase Salmonella growth. There are three reasons: first, it can could reduce the influence of the medium on the conductivity and dielectric characteristics of the entire solution or it would cause difficulties of DEP absorption of Salmonella, and also likely to influence the capacitive impedance measurement; Secondly, it can reduce the demand for preparing the culture medium; Thirdly, could possibly the most important influential factor, the growing surface of proliferating Salmonella is too big for antibody-coated gold-nanoparticles to bind with so the dielectrophoresis force cannot adsorb them to the comb-shape electrodes. Therefore, their capacitance value can not be effectively measured and the real growth number is incalculable. Besides, the excessive proliferation may make measurements beyond saturation and judgment also difficult.

Case (b) Bacteriostatic Antibiotics

Add a certain amount of bacteriostatic antibiotic to inhibit Salmonella survive. Meanwhile, one may optionally add culture medium and antibody-coated gold nanoparticles, and adjust operating frequency and voltage of dielectrophoresis to allow Salmonella to be adsorbed on the electrode with a sufficient DEP force. Measure the impedance value and wait for a certain period of time to allow antibiotic reaction to occur, and then measure the impedance value again. Compare the measured results with those without addition of antibiotic, if the numerical trend is nearly unchanged, it indicates that the antibiotic is competent to inhibit Salmonella. On the contrary, the antibiotic cannot inhibit Salmonella if the numerical reading increases. In this way, the required dosage of the antibiotic and the antibiotic resistance of Salmonella can be detected. Hereby, we could consider excessive proliferation may make measurements beyond saturation that results in a difficult judgment.

### EMBODIMENT 3

As human blood red cells and platelets have no human DNA, white blood cell is the only human cell with the DNA in the blood. The sum of the number of red blood cells and platelets (5,000,000/μL) is one thousand times more than the number of white blood cell (5,000˜10,000/μL). Therefore, using solubilization kits to break down the cell membrane directly from the blood sample is difficult to distinguish different cells. Lysis of cells in different species often makes it indistinguishable between bacterial DNA and host DNA. Furthermore, in DNA analysis for genetic or infectious diseases, bacteria and human cells would be lysed simultaneously if no precedent separation between them. Then DNAs from bacteria and hosts may coexist that would make it difficult for judgment and detection.

In this invention, certain surface protein of white blood cell is utilized to specifically bind to a particular protein with gold-nanoparticles. By only adding the protein-coated nano-particles to the whole blood sample, this method can directly implement the isolation and complete further statistics of WBC number. Furthermore, it can facilitate cell lysis and the processing of DNA, such as DNA sequencing.

In summary, these embodiments of bio-particles (pathogens) detection method demonstrated that the chip and method can directly and effectively separate target bio-particles from the other constituents in the sample, and further measure the amount of target biological particles on the chip. Although the embodiments mainly focus on Salmonella, the invention can not only apply to bacteria but also other pathogens or biological particles as long as their corresponding antibodies or binding proteins are available.

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. A method for detecting bio-particles characterized by the use of a chip having comb-shaped electrodes and at least one well set on said electrodes, said method comprising:
a) adding some sample to the conductive fluid in the test tube, and mixing antibodies-coated metal nanoparticles with bio-particles until the antibodies-coated metal nanoparticles fully integrate with bio-particles;
b) drawing the mixture and dropping in the well of said chip;
c) applying signals of specific frequency to said comb-shaped electrodes, or further heating the bottom of said chip to cause mild convection in the solution, which leads to increasing adsorption opportunity between the gold-nanoparticles modified bio-particles and comb-shaped electrodes;
d) taking out the supernatant mixture and adding water to rinse and keep purified bio-particles adsorbing on the edge of comb-shape electrodes while still applying a specific frequency AC signal to the comb-shaped electrodes to continue adsorbing said modified bio-particles; and
e) measuring impedance between the electrodes, particularly the capacitance, and comparing with the impedance of blank comb-shaped electrodes to conclude that the differences in measured values reflect bio-particles quantity adsorbed on the electrodes.

2. The method of claim 1 wherein said bio-particles mean viruses, bacteria and other cells with surface antigens or proteins to bond the corresponding antibodies or proteins.

3. The method of claim 1 wherein said metal nanoparticles means nano-particles of conductive material.

4. A method for detecting bio-particles characterized by the use of a chip having non-magnetic comb-shaped electrodes and at least one well set on said electrodes, said method comprising:
a) mixing samples and antibodies-coated nano-magnetic beads which are specific to the target bio-particles in said samples;
b) drawing a fixed amount of mixture, and dropping in the well on the chip; using external magnetic field to adsorb and focus the bio-particles modified by antibodies coated nano-magnetic beads on the chip;
c) taking out the supernatant mixture and adding water repeatedly to purify bio-particles, meanwhile the external magnetic field remaining to continue bonding said modified bio-particles on the chip;
d) turning off the external magnetic field, and applying a specific frequency AC signal to said comb-shaped electrode to continue adsorbing said modified bio-particles; and
e) measuring the impedance between the electrodes, particularly the capacitance, and comparing with the impedance of blank comb-shaped electrodes to conclude that the differences in measured values reflect bio-particles quantity adsorbed on the electrodes.

5. The method of claim 4 wherein the bio-particles mean viruses, bacteria and other cells with surface antigens or proteins to bond the corresponding antibodies or proteins.

6. A method for detecting antibiotic-resistance of pathogens by using a chip having non-magnetic comb-shaped electrodes and at least one well set on said electrodes, said method including implementation steps of:
a) adding some sample to the conductive fluid in the test tube, and mixing antibodies-coated metal nanoparticles with pathogens until the antibodies-coated metal nanoparticles fully integrate with pathogens;
b) drawing the mixture and dropping in the well of said chip;
c) applying signals of specific frequency to said comb-shaped electrodes, or further heating the bottom of the chip to cause mild convection in the solution, which leads to increasing adsorption opportunity between said modified pathogens and comb-shaped electrodes;
d) taking out the supernatant mixture and adding water to rinse and keep purified pathogen adsorbing on the edges of comb-shape electrodes while still applying a specific frequency AC signal to said comb-shaped electrodes to continue adsorbing modified pathogens;
e) measuring impedance between the electrodes, particularly the capacitance, and comparing with the blank comb-shaped electrode, the differences in measured values reflect pathogens quantity adsorbed on the electrodes;
f) adding a certain amount of bactericidal antibiotics to reduce pathogen-survival or bacteriostatic antibiotics to inhibit pathogen survive, and measuring the present impedance value;
g) waiting for a certain period of time to let antibiotic reaction occur sufficiently, and again measuring the impedance value; and
h) comparing the measured results of step (g) with those of step (f), which can assist to detect the required amount of antibiotics and the antibiotic-resistance of pathogen.

7. The method of claim 6, wherein said metal nanoparticles mean the nanoparticles of conductive materials.

8. The method of claim 6, wherein said metal nanoparticles mean the nano-magnetic beads with conductivity and magnetism.

9. The method of claim 8, wherein said nano-magnetic beads can be combined with the antibodies and then attached to the target pathogens, which further can be concentrated, purified, and collected by employing external magnetic field.

10. The method of claim 6, wherein step (f) can further include adding some culture medium accompanied by antibiotics.
